(19) United States
(12) Patent Application Publication
(10) Pub. No.: US 2008/0010868 A1
(43) Pub. Date: Jan. 17, 2008

(54) Footwear having cushioning device

(75) Inventor: Li Mei Tsai, Taichung (TW)

Correspondence Address: CHARLES E. BAXLEY, ESQ., 90 JOHN STREET, THIRD FLOOR, NEW YORK, NY, 10038, US

(73) Assignee: Hsin-I Plastic Co., Ltd.

(21) Appl. No.: 11/485,669

(22) Filed: Jul. 13, 2006

(51) Int. Cl. A43B 13/28; A61F 5/14

(52) U.S. Cl. 36/141; 36/27

(57) ABSTRACT

A footwear includes a shoe sole having one or more cavities formed in the upper portion each for receiving a cushioning member, the cushioning member includes a massaging member movable up and down relative to the casing and having an upper portion for engaging with and for massaging a foot of a user, and a spring member received in the cavity of the shoe sole and engaged with the massaging member for biasing and moving the massaging member upwardly beyond the upper portion of the shoe sole to engaging with and to massaging the foot of the user. A casing is engaged into the cavity of the shoe sole and having a chamber for receiving the spring member.

### BACKGROUND OF THE INVENTION

1. Field of the Invention

The present invention relates to a footwear, and more particularly to a footwear including a spring or resilient or cushioning device for softly or resiliently engaging with the feet of the users and for smoothly massaging the feet of the users and for preventing the user from feel pain.

2. Description of the Prior Art

Typical footwear or shoes comprise a shoe sole for supporting the feet of a user, and a shoe upper for engaging with and for retaining the feet of the user in engagement with the shoe sole. For massaging the feet of the users, some of the shoes may provide a massage structure on the shoe sole for engaging with and for massaging the feet of the users.

For example, U.S. Pat. No. 3,589,037 to Gallagher discloses one of the typical foot cushioning support members constructed from a pair of laminated gas impervious sheets of thin, lightweight, elastic material, and having a multiplicity of separate gas filled pockets distributed over the support surface of the member for engaging with and for massaging the feet of the users.

However, when the gas is fully filled within the pockets, the users may feel pain when stepping onto the gas fully filled pockets. On the contrary, when the gas is not fully filled within the pockets, or when the pockets are inflated, the pockets may not be used to suitably massage the feet of the users.

U.S. Pat. No. 4,598,484 to Ma discloses another typical footwear comprising a number of non-slip profiles adjustably connected to the insole for engaging with and for massaging the feet of the users. However, the non-slip profiles have no cushioning structures such that the users may feel pain when stepping onto the non-slip profiles.

The present invention has arisen to mitigate and/or obviate the afore-described disadvantages of the conventional shoes.

### SUMMARY OF THE INVENTION

The primary objective of the present invention is to provide a footwear including a spring or resilient or cushioning device for softly or resiliently engaging with the feet of the users and for smoothly massaging the feet of the users and for preventing the user from feel pain.

In accordance with one aspect of the invention, there is provided a footwear comprising a shoe sole including an upper portion having at least one cavity formed therein, and at least one cushioning member receiving in the cavity of the shoe sole, and including a massaging member movable up and down relative to the casing and having an upper portion for engaging with and for massaging a foot of a user, and a spring member received in the cavity of the shoe sole and engaged with the massaging member for biasing and moving the massaging member upwardly beyond the upper portion of the shoe sole to engaging with and to massaging the foot of the user.

The cushioning member includes a casing engaged into the cavity of the shoe sole and having a chamber formed therein and defined by a peripheral wall for receiving the spring member. The shoe sole includes a latch groove formed in the cavity thereof and communicating with the cavity thereof, and the casing includes a catch extended therefrom for engaging with the latch groove of the shoe sole and for latching and anchoring or positioning the casing to the shoe sole.

The casing includes at least one channel formed therein, and the cushioning member includes at least one projection extended therefrom for engaging into the channel of the casing and for guiding and for limiting the massaging member to move relative to the casing. The upper portion of the massaging member is a curved structure for smoothly and comfortably engaging with the foot of the user and for preventing the user from feel pain.

The shoe sole includes two straps each having a fastening device disposed thereon, the fastening devices of the straps are adjustably engaged with each other for stably retain the foot of the user in engagement with the upper portion of the massaging member. The fastening devices are preferably the hook and loop fastening devices.

Further objectives and advantages of the present invention will become apparent from a careful reading of the detailed description provided hereinbelow, with appropriate reference to the accompanying drawings.

### BRIEF DESCRIPTION OF THE DRAWINGS

FIG. 1 is a perspective view of a footwear in accordance with the present invention;

FIG. 2 is a partial exploded view of the footwear;

FIG. 3 is a top plan schematic view of the footwear;

FIG. 4 is a cross sectional view of the footwear taken along lines 4-4 of FIG. 3;

FIG. 5 is a cross sectional view similar to FIG. 4 illustrating the operation of the footwear;

FIG. 6 is an enlarged partial cross sectional view of the footwear;

FIG. 7 is a perspective view illustrating one of the cushioning members of the footwear; and

FIG. 8 is a perspective view similar to FIG. 1 illustrating the operation of the footwear.

### DETAILED DESCRIPTION OF THE PREFERRED EMBODIMENT

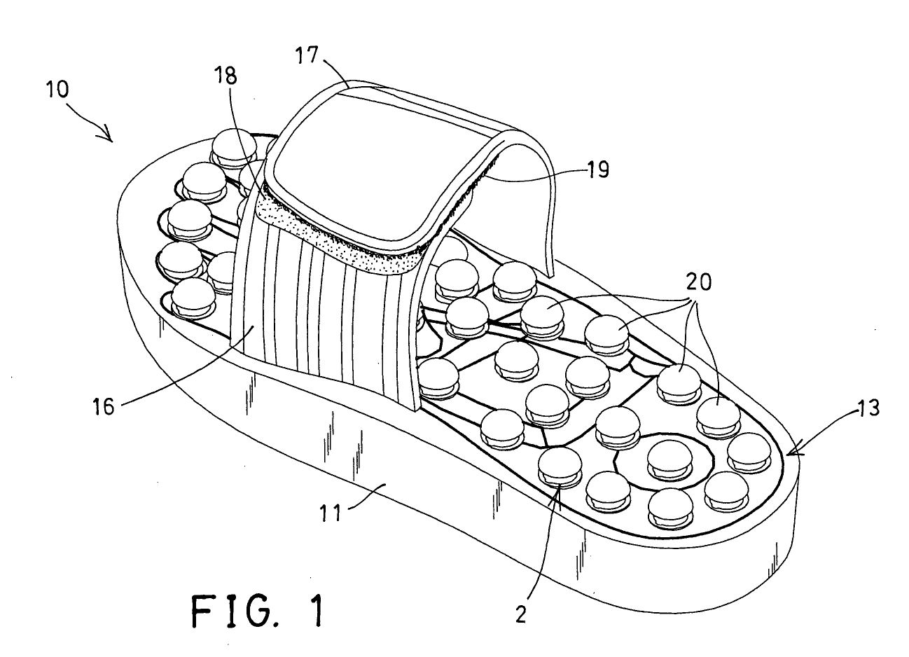

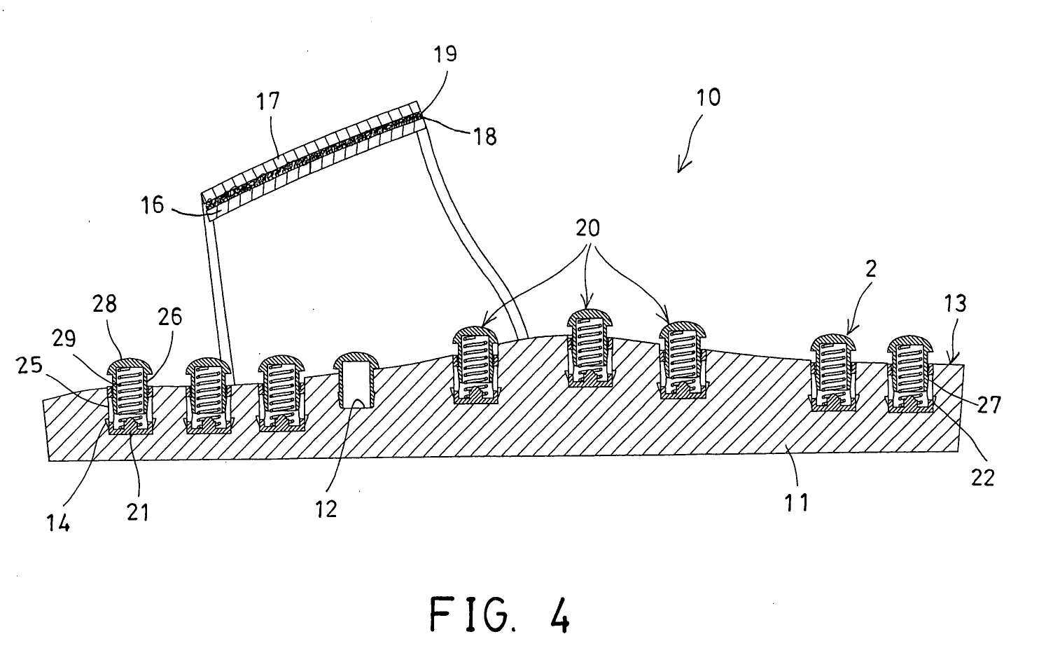

Referring to the drawings, and initially to FIGS. 1-4, a footwear 10 in accordance with the present invention comprises an insole or shoe sole 11 including one or more cavities 12 formed in the upper portion 13 thereof and opened or facing upwardly each for receiving one of the cushioning members 20 of a cushioning device 2 for the footwear 10. The shoe sole 11 includes a peripheral latch groove 14 formed in each of the cavities 12 thereof and communicating with the cavities 12 thereof (FIGS. 4-6). The cushioning device 2 is disposed or provided on the upper portion 13 of the shoe sole 11 for softly or resiliently engaging with and for smoothly massaging the feet of the users.

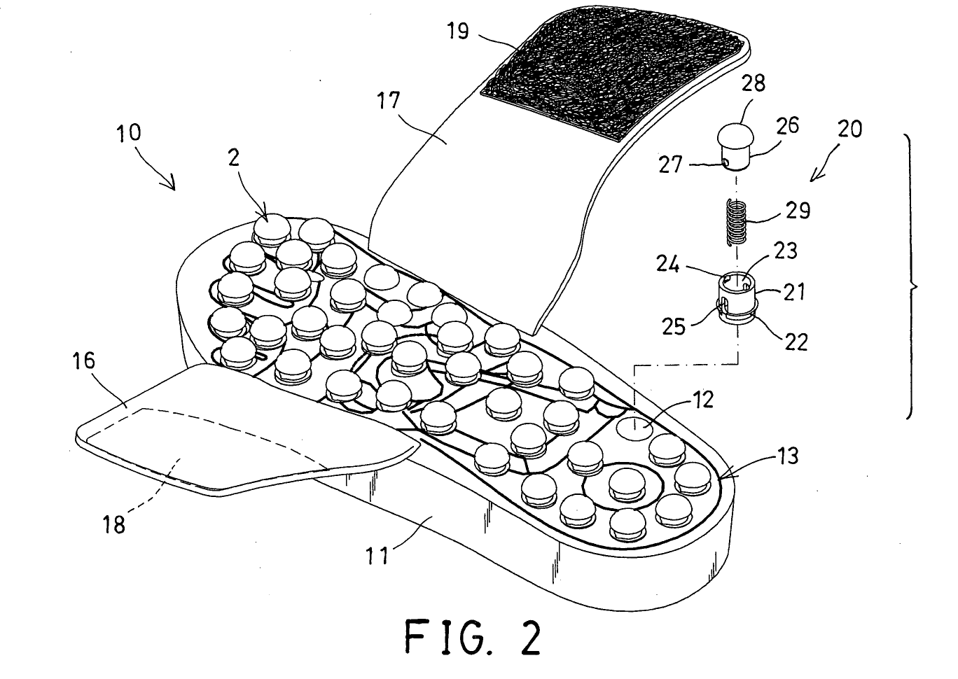

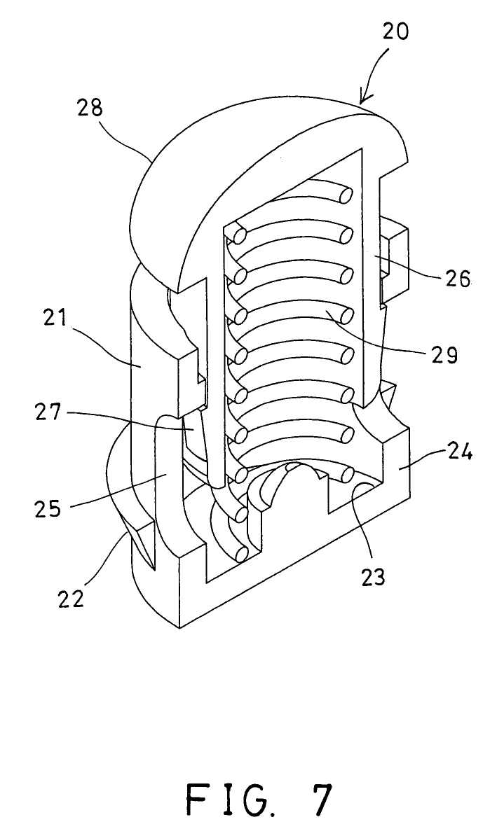

As shown in FIGS. 2 and 4-7, the cushioning members 20 of the cushioning device 2 each include a casing 21 to be engaged into the corresponding cavities 12 of the shoe sole 11, and the casing 21 includes a peripheral catch 22 extended outwardly therefrom for engaging with the corresponding peripheral latch grooves 14 of the shoe sole 11 and for latching or anchoring or positioning the casing 21 to the shoe sole 11, and includes a chamber 23 formed therein and defined by a peripheral wall 24, and includes one or more, such as two oblong holes or channels 25 formed therein, such as oppositely and vertically formed in the peripheral wall 24 and parallel to each other and communicating with the chamber 23 of the casing 21.

The cushioning members 20 of the cushioning device 2 each further include a follower or profile or massaging member 26 slidably engaged into the chamber 23 of the casing 21 and movable up and down relative to the casing 21, and the massaging member 26 includes one or more, such as two projections 27 laterally extended outwardly therefrom for engaging with the corresponding channels 25 of the casing 21 and for guiding the massaging member 26 to move up and down relative to the casing 21, and also for limiting the massaging member 26 to move relative to the casing 21. The massaging member 26 includes an enlarged and smooth or curved outer peripheral surface or upper portion 28 having a curved structure for smoothly and comfortably engaging with and for softly or resiliently massaging the feet of the users.

A spring biasing means or member 29 is received in each of the cavities 12 of the shoe sole 11, or is disposed or engaged into the chamber 23 of the casing 21 and engaged between the massaging member 26 and the casing 21 for biasing the massaging member 26 to move upwardly beyond the casing 21 and/or the upper portion 13 of the shoe sole 11, and thus for allowing the massaging member 26 to suitably or softly or resiliently engage with and to massage the feet of the users. As best shown in FIGS. 4-6, it is preferable that the peripheral wall 24 of the casing 21 includes an upper portion flush with or slightly lower than the upper portion 13 of the shoe sole 11 for allowing the casing 21 to be completely or fully engaged into the cavities 12 of the shoe sole 11, and the massaging members 26 are arranged to be slightly extended upwardly beyond the upper portion 13 of the shoe sole 11 for allowing the massaging member 26 to suitably or softly or resiliently engage with and to massage the feet of the users.

It is to be noted that the curved upper portion 28 may be slightly received or engaged into the corresponding cavities 12 of the shoe sole 11, but may also be slightly extended upwardly beyond the upper portion 13 of the shoe sole 11 for suitably or softly or resiliently engaging with and massaging the feet of the users. It is preferable that the enlarged upper portion 28 of the massaging member 26 includes an outer diameter greater than the inner diameter of the chamber 23 of the casing 21 for allowing the enlarged upper portion 28 of the massaging member 26 to be engaged with the casing 21 and for preventing the massaging member 26 from being completely engaged into the chamber 23 of the casing 21.

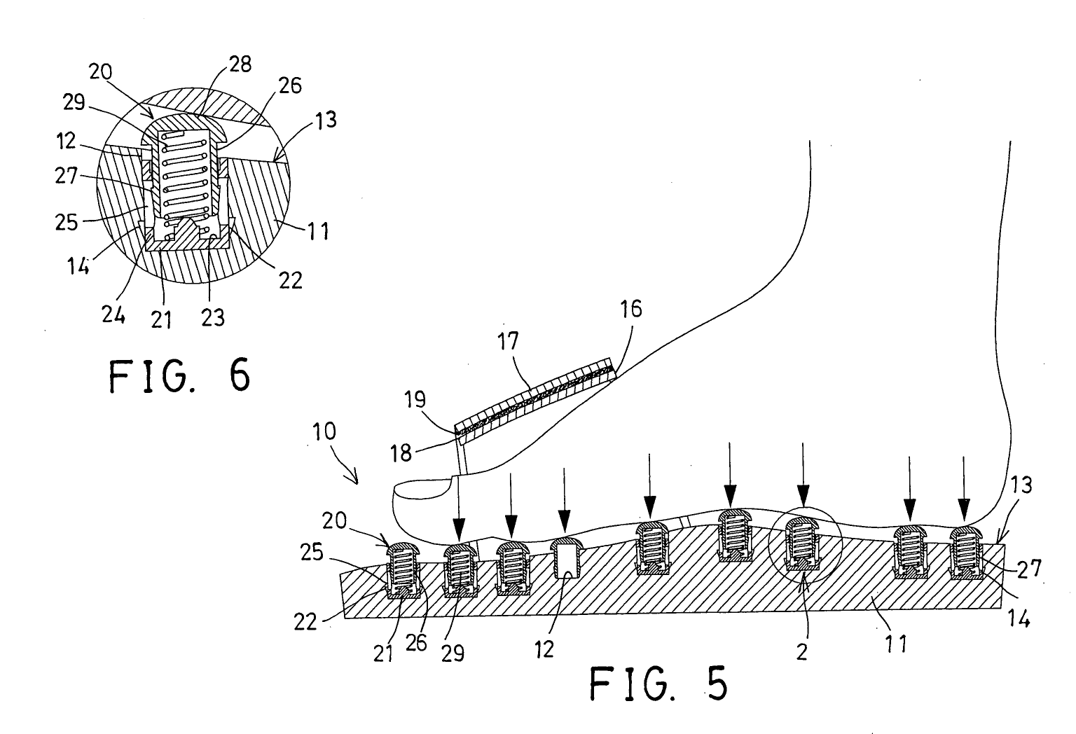

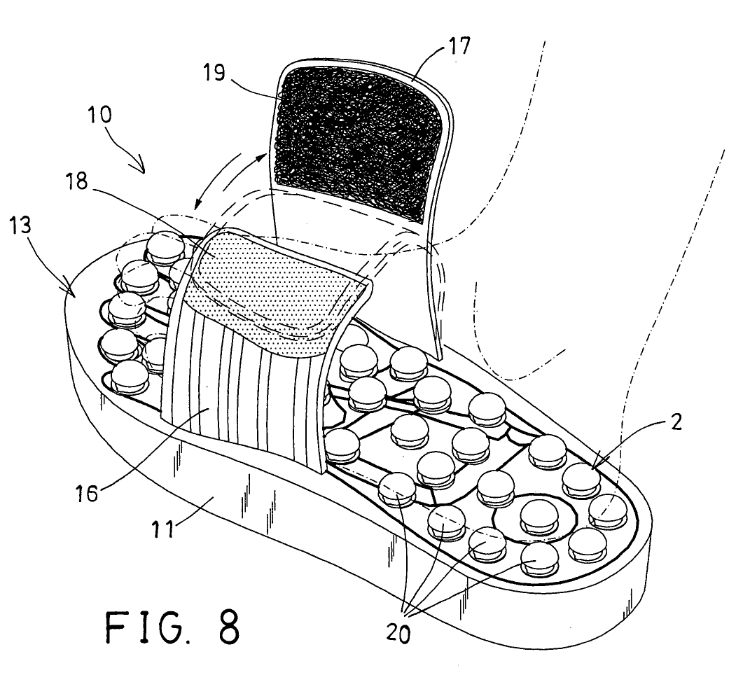

In operation, as shown in FIGS. 5 and 6, the feet of the users may be contacted and engaged onto the curved upper portions 28 of the massaging members 26 against the spring biasing force of the spring members 29, and the spring members 29 may bias the massaging members 26 to suitably or softly or resiliently engage with and to massage the feet of the users, and to prevent the users from feeling pain when stepping onto the spring biased massaging members 26. The footwear 10 further includes one or more, such as two side straps 16, 17 each having a fastening device 18, 19 disposed or provided thereon, such as the hook and loop fastening device, and the fastening devices 18, 19 of the side straps 16, 17 may be adjustably engaged with each other (FIGS. 1-5 and 8) for stably retain the feet of the users in engagement with the shoe sole 11 and/or the curved upper portions 28 of the massaging members 26.

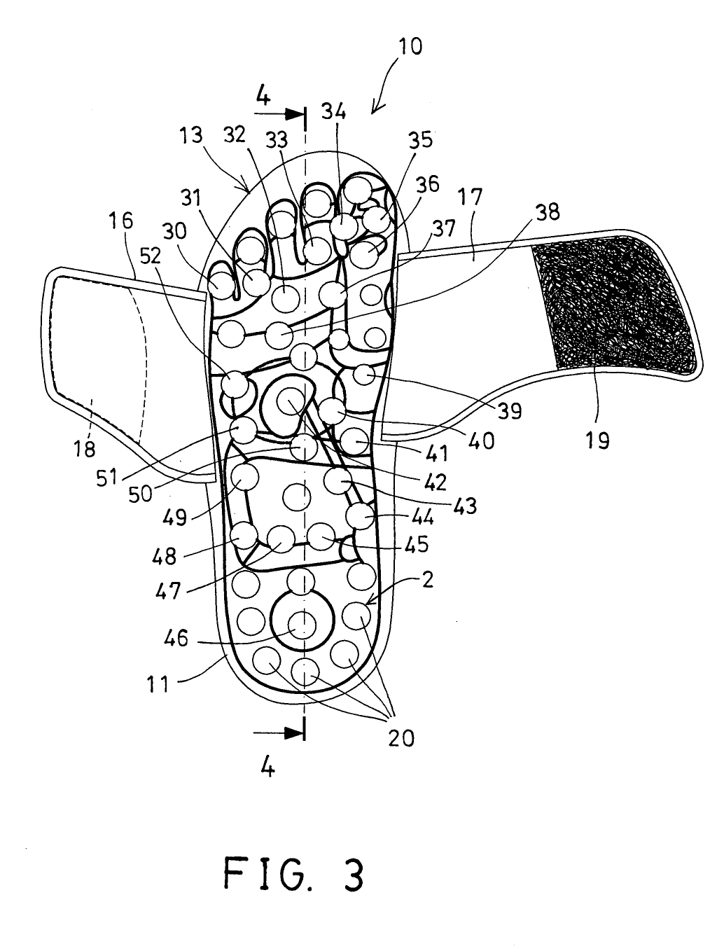

As shown in FIG. 3, our feet include many areas corresponding or reactive to various kinds of internal organs, for example, the area 30 is the reactive area for ears, the area 31 is for the frontal sinus, the area 32 is for the head and the shoulder portions of the users, the area 33 is for the eyes, the area 34 is for the temple, the area 35 is for the noses, the area 36 is for the pituitary gland, the area 37 is for the parathyroid gland, the area 38 is for the lungs and the trachea, the area 39 is for the stomach, the area 40 is for the duodenum, the area 41 is for the pancreas, the area 42 is for the kidney, the area 43 is for the ureter, the area 44 is for the anus, the area 45 is for the bladder, the area 46 is for the testis and the ovary, the area 47 is for the small intestine, the area 48 is for the rectum, the area 49 is for the descending colon, the area 50 is for the transverse colon, the area 51 is for the spleen, the area 52 is for the heart, etc.

When some of the areas 30-52 are not required to be massaged, the corresponding cushioning members 20 of the cushioning device 2 may be removed, and the required cushioning members 20 of the cushioning device 2 at the selected areas may be retained in engagement in the corresponding cavities 12 of the shoe sole 11 in order to massage only the required or the selected areas.

Accordingly, the footwear in accordance with the present invention includes a spring or resilient or cushioning device for softly or resiliently engaging with the feet of the users and for smoothly massaging the feet of the users and for preventing the user from feel pain.

Although this invention has been described with a certain degree of particularity, it is to be understood that the present disclosure has been made by way of example only and that numerous changes in the detailed construction and the combination and arrangement of parts may be resorted to without departing from the spirit and scope of the invention as hereinafter claimed.

I claim:

1. A footwear comprising:
a shoe sole including an upper portion having at least one cavity formed therein, and
at least one cushioning member receiving in said at least one cavity of said shoe sole, and including a massaging member movable up and down relative to said casing and having an upper portion for engaging with and for massaging a foot of a user, and a spring member received in said at least one cavity of said shoe sole and engaged with said massaging member for biasing and moving said massaging member upwardly beyond said upper portion of said shoe sole to engaging with and to massaging the foot of the user.

2. The footwear as claimed in claim 1, wherein said at least one cushioning member includes a casing engaged into said at least one cavity of said shoe sole and having a chamber formed therein and defined by a peripheral wall for receiving said spring member.

3. The footwear as claimed in claim 2, wherein said shoe sole includes a latch groove formed in said at least one cavity thereof and communicating with said at least one cavity thereof, and said casing includes a catch extended therefrom for engaging with said latch groove of said shoe sole and for latching and anchoring said casing to said shoe sole.

4. The footwear as claimed in claim 2, wherein said casing includes at least one channel formed therein, and said at least one cushioning member includes at least one projection extended therefrom for engaging into said at least one channel of said casing and for guiding and for limiting said massaging member to move relative to said casing.

5. The footwear as claimed in claim 1, wherein said upper portion of said massaging member is a curved structure for smoothly and comfortably engaging with the foot of the user.

6. The footwear as claimed in claim 1, wherein said shoe sole includes two straps each having a fastening device disposed thereon, said fastening devices of said straps are adjustably engaged with each other for stably retain said foot of said user in engagement with said upper portion of said massaging member.

7. The footwear as claimed in claim 6, wherein said fastening devices are hook and loop fastening devices.
